(19) United States
(12) Patent Application Publication
(10) Pub. No.: US 2020/0340058 A1
(43) Pub. Date: Oct. 29, 2020

(54) COMPOSITIONS AND METHODS FOR ENHANCING CELL TRANSPLANTATION EFFICACY

(71) Applicant: THE CHILDREN'S MEDICAL CENTER CORPORATION, BOSTON, MA (US)

(72) Inventors: ANGELA LEK, BOSTON, MA (US); LOUIS M. KUNKEL, BOSTON, MA (US); JANELLE M. SPINAZZOLA, BOSTON, MA (US); YUANFAN ZHANG, BOSTON, MA (US)

(73) Assignee: THE CHILDREN'S MEDICAL CENTER CORPORATION, BOSTON, MA (US)

(21) Appl. No.: 16/645,112

(22) PCT Filed: Sep. 12, 2018
(86) PCT No.: PCT/US2018/050709

§ 371 Date: Mar. 6, 2020

Related U.S. Application Data
(60) Provisional application No. 62/558,632, filed on Sep. 14, 2017.

(51) Int. Cl. C12Q 1/6883; C12N 5/077; C12N 9/22; C12N 15/113; C12N 15/10; A61K 35/34

(52) U.S. Cl. CPC C12Q 1/6883; C12N 5/0658; C12N 9/22; C12N 15/113; C12N 2310/20; A61K 35/34; C12N 2510/00; C12N 2800/80; C12N 15/1093

(57) ABSTRACT

The present invention features compositions and methods that enhance cell extravasation and/or muscle cell fusion, and methods for identifying genes that enhance or inhibit extravasation and muscle cell fusion. One aspect of the present disclosure provides an isolated cell lacking a gene of Table 1 or expressing a reduced level of a gene of Table 1. In some embodiments, the isolated cell has a CRISPR-edited genome or expresses an inhibitory nucleic acid molecule targeting a gene of Table 1. In some embodiments, this inhibitory nucleic acid molecule is an antisense oligonucleotide molecule, a short interfering RNA (siRNA) molecule, or an small hairpin (shRNA) molecule. Another aspect of the present disclosure provides an isolated cell comprising an expression vector encoding a gene of Table 1.

### CROSS-REFERENCE TO RELATED APPLICATION

This application claims the benefit of the following U.S. Provisional Application No. 62/558,632, filed Sep. 14, 2017, the entire contents of which are incorporated herein by reference.

### BACKGROUND OF THE INVENTION

The muscular dystrophies are incurable diseases for which cell-based therapies present a promising therapeutic option. Together they represent a heterogeneous group of genetic disorders that result in progressive degeneration of all muscles in the body. While the root cause of all muscular dystrophies occurs on a genetic level, gene therapy alone is unable to reverse existing muscle degeneration, particularly in aged patients for which body-wide muscle wasting is pervasive. Complete reversal of muscle damage requires replacement of diseased muscles with new healthy muscles, potentially achievable via transplant of muscle stem cells. Progress in the field has stalled for many years due to the low rate of cell extravasation from the circulatory system involved in systemic delivery and the inability for cells to fuse with existing muscle fibers. This disclosure is directed to overcoming these challenges and other important needs.

### SUMMARY OF THE INVENTION

As described below, the present invention features compositions and methods that enhance cell extravasation and/or muscle cell fusion, and methods for identifying genes that enhance or inhibit extravasation and muscle cell fusion.

One aspect of the present disclosure provides an isolated cell lacking a gene of Table 1 or expressing a reduced level of a gene of Table 1. In some embodiments, the isolated cell has a CRISPR (clustered regularly interspaced short palindromic repeats)-edited genome or expresses an inhibitory nucleic acid molecule targeting a gene of Table 1. In some embodiments, this inhibitory nucleic acid molecule is an antisense oligonucleotide molecule, a short interfering RNA (siRNA) molecule, or an small hairpin (shRNA) molecule.

Another aspect of the present disclosure provides an isolated cell comprising an expression vector encoding a gene of Table 1.

In some embodiments of either aspect described above, the isolated cell is a genetically engineered cell derived from a subject in need of cell transplantation therapy. In some embodiments, the cell is a healthy cell. In still other embodiments, the cell is a muscle cell or a cancer cell.

Provided herein are pharmaceutical compositions comprising a muscle cell or a cancer cell lacking a gene of Table 1 or expressing a reduced level of a gene of Table 1 or comprising an expression vector encoding a gene of Table 1. In some embodiments, the pharmaceutical composition also includes a pharmaceutically acceptable excipient.

Other aspects of the present disclosure provide methods for treating a subject in need of muscle cell transplantation, the method comprising administering to the subject a muscle cell lacking a gene of Table 1 or expressing a reduced level of a gene of Table 1 or comprising an expression vector encoding a gene of Table 1.

In yet another aspect of the present disclosure, methods are provided for identifying a gene required for cell fusion, wherein the method involves editing each gene in a genome of a cell population using a clustered regularly interspaced short palindromic repeats (CRISPR) library. The cell population is then grown under conditions that permit cell fusion, and mononucleate cells that are fusion defective are isolated. sgRNAs that are enriched in fusion defective cells are then identified. In some embodiments, the library is a knock-out or upregulation library.

Another aspect provides methods for identifying a gene that promotes extravasation, the method comprising editing each gene in a genome of a cell population using a clustered regularly interspaced short palindromic repeats (CRISPR) library, wherein each cell of the cell population comprises a detectable reporter; administering the cell population to a non-human mammal; isolating cells comprising the detectable reporter from the mammal; and sequencing the genomes of the cells to identify the CRISPR gene edit in each cell, thereby identifying a gene required for extravasation. In some embodiments, the library is a knock-out or upregulation library.

In another aspect, the invention provides a method is also provided for identifying an agent that mimics the effect of an edited gene, wherein the method includes performing the method for identifying a gene required for cell fusion or performing the method for identify a gene that promotes extravasation in the presence of the agent.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal number or function of a cell, tissue, or organ. Such diseases include muscular disorders that disrupt the function or number of muscle cells. Exemplary muscular disorders include muscular dystrophy (Limb-girdles, Facioscapulohumeral dystrophy, Duchenne, Becker's) and age-related muscle-wasting (sarcopenia). Cancer is a disease that involves the inappropriate proliferation and often the metastasis of cancer cells. During the metastasis process the cells undergo extravasation. Accordingly, genes that are required for extravasation could be disrupted in cancer cells (e.g., knockout or reduced using an inhibitory nucleic acid molecule), thereby reducing metastasis.

By “effective amount” is meant the amount of a cell of the invention required to ameliorate the symptoms of a disease relative to an untreated patient. In one embodiment, a cell of the invention comprises a CRISPR edited gene or over-expresses a gene described in Table 1. The effective amount of a muscle cell of the invention used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets (e.g., genes listed in Table 1 and their encoded proteins) that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art.

Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1: Therapeutic strategy for autologous cell transplantation.

FIGS. 2A and 2B are schematic diagrams showing approaches to gene knock-out and activation in forward genetic screens. FIG. 2A is a schematic of gene knockout and activation using Cas9. Gene knock-out is achieved by Cas9-induced cleavage and indel formation at target genomic sites complementary to the sgRNA sequence (left). Gene activation is achieved by inactivated Cas9 and fusion of activation domains (VP64, p65, HSF1) to recruit transcriptional machinery to transcriptional start sites complementary to the sgRNA sequence (right). FIG. 2B shows a screening strategy for genome-wide CRISPR loss and gain-of-function screens.

FIGS. 3A-3C provide results from a fusion screen. FIG. 3A is a micrograph showing a gene-edited myogenic line cultured for six days in differentiation medium. Cells have either fused to form multinuclear myotubes or are fusion defective and remain as mononuclear myoblasts (arrows). FIG. 3B shows isolated mononuclear myoblast population after filtration of mixed population shown in FIG. A. FIG. 3C provides a list of sgRNAs that were significantly enriched in fusion-defective myoblasts (two independent screens). Statistical analysis was performed using MaGeCK software, which takes into account level of guide enrichment and the number of unique guides enriched per gene in order to determine ranking and significance.

FIG. 4 is a schematic diagram showing the strategy of a fusion screen.

FIG. 5 is a schematic diagram showing the strategy of an engraftment screen. FIG. 5A is a micrograph of a human myoblast line stably expressing EGFP-Cas9. FIG. 5B is a flow diagram of an experiment to isolate pro-engraftment myogenic cells.

### DETAILED DESCRIPTION OF THE INVENTION

The invention features compositions and methods that enhance cell extravasation and/or muscle cell fusion, and methods for identifying genes that enhance or inhibit extravasation and muscle cell fusion.

### Cell Therapy for Muscular Dystrophy

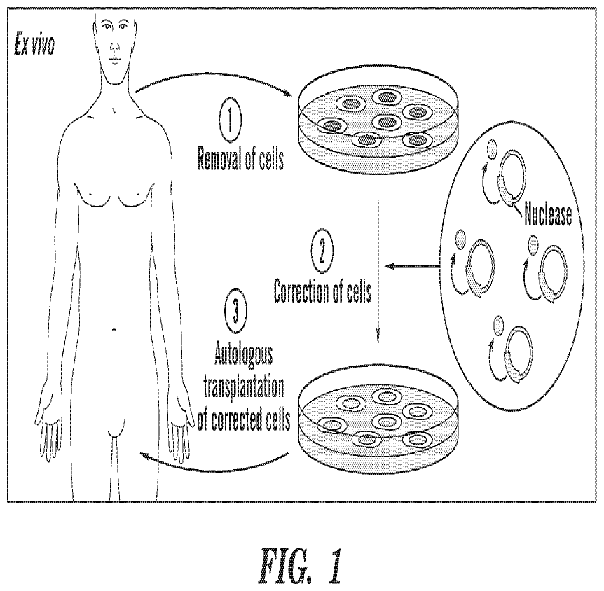

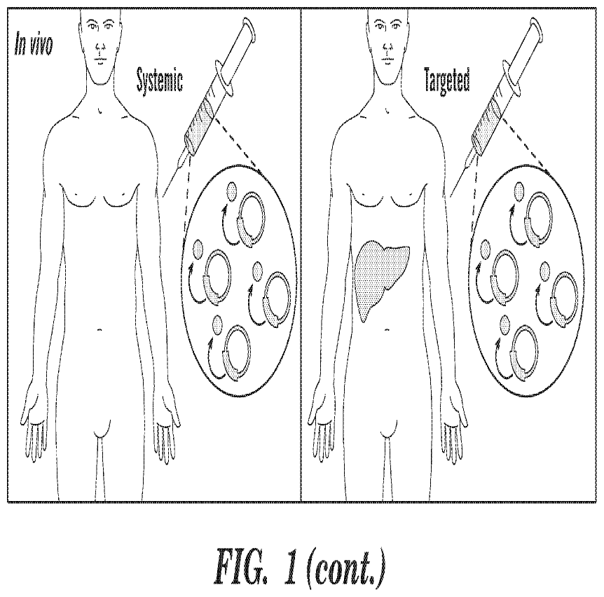

Different strategies for cell therapy have been tested for muscular dystrophy, albeit with limited efficacy to restore skeletal muscle function. See, for example, US Patent Publication No. 20030003085, which is incorporated by reference in its entirety. Skeletal muscles are the most abundant tissue in the human body, thus replacement strategies necessitate large amounts of transplantable cells to achieve therapeutic levels. The body-wide distribution of skeletal muscles also poses a great challenge given that locally transplanted cells do not easily migrate. Intra-arterial delivery of cells presents a viable option to achieve systemic delivery via the circulatory system but is limited to cells that can cross the vessel wall through a process known as ‘extravasation.’ There is currently no process by which corrective cells can be systemically delivered to achieve extravasation and fusion with body-wide musculature. Many different cell types such as satellite cells, myoblasts, muscle derived stem cells, mesoangioblasts, induced pluripotent stem cells, have been trialed for transplantation. Ultimately, the strategy described herein involves autologous cell transplantation (where a patient's own cells are used), utilizing personalized medicine to avoid or remove barriers such as immune rejection of transplanted cells. Generation of muscle stem cells via ‘reprogramming’ of adult cells (from skin or fat) is now widely performed, and combined with CRISPR gene-editing allows for in vitro correction of muscular dystrophy mutations prior to transplantation (FIG. 1). As reported herein, the invention provides the use of human myoblast cells having the genetic characteristics of a transplantable cell population. In one embodiment, the transplant comprises ‘corrected’ patient myogenic stem cells reprogrammed from a pool of induced pluripotent stem cell (iPSC). Accordingly, this disclosure provides a cell replacement therapy to effectively treat muscular dystrophies.

### Cell Transplantation Therapy

Myoblasts are isolated from the skeletal muscle of any mammal according to methods generally known in the art. For example, myoblast samples can be isolated from muscle biopsies using standard culture techniques as described in, for example, Blau, H. M. et al., Adv. Exp. Med. Biol., 280:97-100 (1990); Blau, H. M. et al., Proc. Natl. Acad. Sci. USA, 78:5623-5627 (1981); and Rando, T. A. and Blau, H. M., J. Cell Biol., 125:1275-1287 (1994), the teachings of which are incorporated herein by reference. See also, e.g., Webster, C. et al., Exp. Cell Res., 174:252-265 (1988); Gussoni, E. et al., Nature, 356:435-438 (1992); Karpati, G. et al., Ann. Neurol., 34:8-17 (1993); Walsh, F. A. et al., Adv. Exp. Med. Biol., 28:41-46 (1990); Ham, R. G. et al., Adv. Exp. Med. Biol., 280:193-199 (1990); and Morgan, J. E. et al., J. Neurol. Sci., 86:137-147 (1988). Myoblast samples used in the muscle stem cell purification and separation methods described in US Patent Publication No. 20030003085 typically comprise about 10⁴ to 10⁸ cells, and preferably, about 10⁶ cells. Myoblasts samples used in the muscle stem cell purification and separation methods of the present invention can also comprise more than 10⁸ cells.

Myoblasts of the invention have the ability to extravasate and fuse into host muscle. Specifically, by injecting 10,000-20,000 muscle cells into circulation, a larger percentage of these cells fuse with the host muscle than do normal control muscle cells. In another embodiment, myoblasts of the invention are administered by intramuscular injection (Karpati, G. et al., Am. J. Pathol., 135:27-32 (1989); Fan, Y. et al., Muscle Nerve, 19:853-860 (1996); and Beauchamp, J. et al., Muscle Nerve, Supplement 1: S261 (1994)).

In one embodiment, an effective amount of myoblast cells is transplanted into a mammal in need of such treatment (also referred to as a “recipient” or a “recipient mammal”). As used herein, “donor” refers to a mammal that is the natural source of the cells. In one embodiment, the donor is a healthy mammal (e.g., a mammal that is not suffering from a muscle disease or disorder). In a particular embodiment, the donor and recipient are matched for immunocompatibility. Preferably, the donor and the recipient are matched for their compatibility for the major histocompatibility complex (MHC) (human leukocyte antigen (HLA)) class I (e.g., loci A, B, C) and class II (e.g., loci DR, DQ, DRW) antigens. Immunocompatibility between donor and recipient is determined according to methods generally known in the art (see, e.g., Charron, D. J., Curr. Opin. Hematol., 3:416-422 (1996); Goldman, J., Curr. Opin. Hematol., 5:417-418 (1998); and Boisjoly, H. M. et al., Opthalmology, 93:1290-1297 (1986)). In an embodiment of particular interest, the recipient is a human patient. In another embodiment, a myoblast cell suitable for transplantation is derived from the donor and genetically engineered to correct a genetic defect or CRISPR edited.

As used herein, muscle diseases and disorders include, but are not limited to, recessive or inherited myopathies, such as, but not limited to, muscular dystrophies. Muscular dystrophies are genetic diseases characterized by progressive weakness and degeneration of the skeletal or voluntary muscles which control movement. The muscles of the heart and some other involuntary muscles are also affected in some forms of muscular dystrophy. The histology associated with these diseases is characterized by variation in fiber size, muscle cell necrosis and regeneration, and often proliferation of connective and adipose tissue. Muscular dystrophies are described in the art and include Duchenne muscular dystrophy (DMD), Becker muscular dystrophy (BMD), myotonic dystrophy (also known as Steinert's disease), limb-girdle muscular dystrophies, facioscapulohumeral muscular dystrophy (FSH), congenital muscular dystrophies, oculopharyngeal muscular dystrophy (OPMD), distal muscular dystrophies, and Emery-Dreifuss muscular dystrophy. See, e.g., Hoffman et al., N. Engl. J. Med., 318:1363-1368 (1988); Bonnemann, C. G. et al., Curr. Opin. Ped., 8:569-582 (1996); Worton, R., Science, 270:755-756 (1995); Funakoshi, M. et al., Neuromuscul. Disord., 9(2):108-114 (1999); Lim, L. E. and Campbell, K. P., Curr. Opin. Neurol., 11(5):443-452 (1998); Voit, T., Brain Dev., 20(2):65-74 (1998); Brown, R. H., Annu. Rev. Med., 48:457-466 (1997); Fisher, J. and Upadhyaya, M., Neuromuscul. Disord., 7(1):55-62 (1997), each of which are incorporated entirely incorporated herein by reference.

Two major types of muscular dystrophy, DMD and BMD, are allelic, lethal degenerative muscle diseases. DMD results from mutations in the dystrophin gene on the X chromosome (Hoffman et al., N. Engl. J. Med., 318:1363-1368 (1988)), which usually result in the absence of dystrophin, a cytoskeletal protein in skeletal and cardiac muscle. BMD is the result of mutations in the same gene (Hoffman et al., N. Engl. J. Med., 318:1363-1368 (1988)), but with dystrophin usually expressed in muscle at a reduced level and/or as a shorter, internally deleted form, resulting in a milder phenotype.

Thus, the present disclosure also provides a method of treating a muscle disease or disorder in a mammal in need thereof comprising administering an effective amount of donor muscle cells to the mammal. One embodiment of the disclosurerelates to a method of treating a muscular dystrophy in a mammal in need thereof comprising administering an effective amount of donor muscle cells to the mammal. In another embodiment, the disclosure provide a method of treating DMD in a mammal in need thereof comprising administering an effective amount of donor cells to the mammal. Another embodiment provides a method of treating BMD in a mammal in need thereof comprising administering an effective amount of donor muscle cells to the mammal. Desirably, the cells described herein extravasate and fuse with DMD or BMD host muscle fibers, contributing dystrophin-competent myonuclei to the host fibers (mosaic fibers). The expression of normal (donor) dystrophin genes in such fibers can generate sufficient dystrophin to confer a normal phenotype to these muscle fibers.

This disclosure also relates to a method of treating a limb-girdle muscular dystrophy in a mammal in need thereof comprising administering an effective amount of purified donor muscle cells to the mammal.

### Genetically Engineered Myoblasts

Cells generated according to the methods of the present disclosure can also be used in gene therapy, a utility enhanced by the ability of the cells to extravasate and fuse. Cells, as described herein can be genetically altered by one of several means known in the art to comprise functional genes which may be defective or lacking in a mammal requiring such therapy. The recombinant muscle cells can then be transferred to a mammal, wherein they will fuse and, additionally, express recombinant genes. Using this technique, a missing or defective gene in a mammal's muscular system can be replaced or supplemented by infusion of genetically altered muscle cells. Gene therapy applications can use muscle cells as described herein to provide essential gene products through secretion from muscle tissue into the bloodstream (i.e., into circulation). Because muscle cells extravasate and fuse together, they are capable of contributing progeny comprising recombinant genes to multiple multinucleated myofibers during normal muscular development.

Thus, muscle cells purified or isolated in accordance with the methods of the present disclosure can be used for delivery of a desired nucleic acid product to the circulatory system of a mammal (e.g., a human or other mammal or vertebrate). In this method, a nucleic acid sequence encoding a desired nucleic acid product is introduced into muscle cells. Typically, the nucleic acid sequence will be a gene that encodes the desired nucleic acid product. Such a gene is typically operably linked to suitable control sequences capable of effecting the expression of the desired nucleic acid product in muscle cells. The term “operably linked”, as used herein, is defined to mean that the gene (or the nucleic acid sequence) is linked to control sequences in a manner which allows expression of the gene (or the nucleic acid sequence). Generally, operably linked means contiguous.

Control sequences include a transcriptional promoter, an optional operator sequence to control transcription, a sequence encoding suitable mRNA ribosomal binding sites and sequences which control termination of transcription and translation. Suitable control sequences also include myoblast-specific transcriptional control sequences (see, e.g., U.S. Pat. No. 5,681,735, the teachings of which are incorporated herein by reference). Thus, in a particular embodiment, a recombinant gene (or a nucleic acid sequence) encoding a desired nucleic acid product is operably linked to myoblast-specific control sequences capable of effecting the expression of the desired nucleic acid product in muscle cells. In a further embodiment, a nucleic acid sequence encoding a desired nucleic acid product can be placed under the regulatory control of a promoter which can be induced or repressed, thereby offering a greater degree of control with respect to the level of the product in the muscle cells.

As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known in the art. Exemplary promoters include the SV40 and human elongation factor (EFI). Other suitable promoters are readily available in the art (see, e.g., Ausubel et al., Current Protocols in Molecular Biology, John Wiley & Sons, Inc., New York (1998); Sambrook et al., Molecular Cloning: A Laboratory Manual, 2nd edition, Cold Spring Harbor University Press, New York (1989); and U.S. Pat. No. 5,681,735).

Nucleic acid sequences are defined herein as heteropolymers of nucleic acid molecules. The nucleic acid molecules can be double stranded or single stranded and can be a deoxyribonucleic acid (DNA) molecule, such as complementary DNA (cDNA) or genomic DNA, or a ribonucleic acid (RNA) molecule. As such, the nucleic acid sequence can, for example, include one or more exons, with or without, as appropriate, introns, as well as one or more suitable control sequences. In one example, the nucleic acid molecule contains a single open reading frame which encodes a desired nucleic acid product. The nucleic acid sequence is operably linked to a suitable promoter.

A nucleic acid sequence encoding a desired nucleic acid product can be isolated from nature, modified from native sequences, or manufactured de novo, as described in, for example, Ausubel et al., Current Protocols in Molecular Biology, John Wiley & Sons, New York (1998); and Sambrook et al., Molecular Cloning: A Laboratory Manual, 2nd edition, Cold Spring Harbor University Press, New York. (1989). Nucleic acids can be isolated and fused together by methods known in the art, such as exploiting and manufacturing compatible cloning or restriction sites.

As used herein, the term “desired nucleic acid product” refers to a protein, polypeptide, DNA (e.g., genes, antisense DNA), or RNA (e.g., ribozymes) that is expressed from nucleic acid in a mammal. In a particular embodiment, the desired nucleic acid product is a heterologous therapeutic protein. For example, in the treatment of a mammal with DMD or BMD, the desired nucleic acid product can be dystrophin. In the treatment of a mammal with a limb-girdle muscular dystrophy, desired nucleic acid products include, but are not limited to, calpain-3 and sarcoglycan complex members (e.g., .alpha.-sarcoglycan, .beta.-sarcoglycan, .gamma.-sarcoglycan and .delta.-sarcoglycan). In the treatment of a mammal with a congenital muscular dystrophy, desired nucleic acid products include, but are not limited to, laminin alpha 2-chain.

Nucleic acid sequences encoding a desired nucleic acid product can be introduced into purified muscle cells by a variety of methods (e.g., transfection, infection, transformation, direct uptake, projectile bombardment, and liposome-mediated delivery). In a particular embodiment, a nucleic acid sequence encoding a desired nucleic acid product is inserted into a nucleic acid vector, e.g., a DNA plasmid, virus, or other suitable replicon (e.g., viral vector). As a particular example, a nucleic acid sequence encoding a desired nucleic acid product is integrated into the genome of a virus which is subsequently introduced into purified muscle cells. The term “integrated”, as used herein, refers to the insertion of a nucleic acid sequence (e.g., a DNA or RNA sequence) into the genome of a virus as a region which is covalently linked on either side to the native sequences of the virus. Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma virus, mammalian B, C, and D type viruses, human T-cell lymphotrophic virus-bovine leukemia virus (HTLV-BLV) group, lentiviruses, spumaviruses (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, and lentiviruses. Other examples of vectors are described, for example, in McVey et al., U.S. Pat. No. 5,801,030, the teachings of which are incorporated herein by reference.

Packaging cell lines can be used for generating recombinant viral vectors comprising a recombinant genome that includes a nucleotide sequence (e.g., RNA or DNA) encoding a desired nucleic acid product. The use of packaging cell lines can increase both the efficiency and the spectrum of infectivity of the produced recombinant virions.

Packaging cell lines useful for generating recombinant viral vectors comprising a recombinant genome that includes a nucleotide sequence encoding a desired nucleic acid product are produced by transfecting host cells, such as mammalian host cells, with a viral vector including the nucleic acid sequence encoding the desired nucleic acid product integrated into the genome of the virus, as described herein. Suitable host cells for generating cell lines include human (such as HeLa cells), bovine, ovine, porcine, murine (such as embryonic stem cells), rabbit, and monkey (such as COS1 cells) cells. A suitable host cell for generating a cell line may be an embryonic cell, a bone marrow stem cell, or other progenitor cell. Somatic cells contemplated by the present disclosure can be, for example, an epithelial cell, a fibroblast cell, a smooth muscle cell, a blood cell (including a hematopoietic cell, red blood cell, T-cell, B-cell, etc.), a tumor cell, a cardiac muscle cell, a macrophage, a dendritic cell, a neuronal cell (e.g., a glial cell or an astrocyte), or a pathogen-infected cell (e.g., cells infected by bacteria, viruses, virusoids, parasites, or prions). These cells can be obtained commercially or from a depository or obtained directly from an individual, such as by biopsy. Viral stocks are harvested according to methods generally known in the art. See, e.g., Ausubel et al., Eds., Current Protocols in Molecular Biology, John Wiley & Sons, New York (1998); Sambrook et al., Eds., Molecular Cloning: A Laboratory Manual, 2nd edition, Cold Spring Harbor University Press, New York (1989); Danos and Mulligan, U.S. Pat. No. 5,449,614; and Mulligan and Wilson, U.S. Pat. No. 5,460,959, and the teachings of each are incorporated herein by reference.

Examples of suitable methods of transfecting or transforming muscle cells include infection, calcium phosphate precipitation, electroporation, microinjection, lipofection, and direct uptake. Such methods are described in more detail, for example, in Sambrook et al., Molecular Cloning: A Laboratory Manual, Second Edition, Cold Spring Harbor University Press, New York (1989); Ausubel, et al., Current Protocols in Molecular Biology, John Wiley & Sons, New York (1998); and Danos and Mulligan, U.S. Pat. No. 5,449,614, and the teachings of each are incorporated herein by reference.

Virus stocks consisting of recombinant viral vectors comprising a recombinant genome that includes a nucleotide (DNA or RNA) sequence encoding a desired nucleic acid product, are produced by maintaining the transfected cells under conditions suitable for virus production (e.g., in an appropriate growth media and for an appropriate period of time). Such conditions, which are not critical to the invention, are generally known in the art. See, e.g., Sambrook et al., Molecular Cloning: A Laboratory Manual, Second Edition, Cold Spring Harbor University Press, New York (1989); Ausubel et al., Current Protocols in Molecular Biology, John Wiley & Sons, New York (1998); U.S. Pat. No. 5,449,614; and U.S. Pat. No. 5,460,959, and the teachings of each are incorporated herein by reference.

A vector comprising a nucleic acid sequence encoding a desired nucleic acid product can also be introduced into muscle cells by targeting the vector to cell membrane phospholipids. For example, targeting of a vector can be accomplished by linking the vector to a vesicular stomatitis virus-glycoprotein (VSV-G protein), a viral protein with affinity for all cell membrane phospholipids. Such a construct can be produced using methods well-known to those practiced in the art.

As a particular example of the above approach, a recombinant gene (or a nucleic acid sequence) encoding a desired nucleic acid product and operably linked to myoblast-specific control sequences capable of effecting the expression of the desired nucleic acid product in purified muscle cells can be integrated into the genome of a virus that enters the cells. By infecting muscle cells, the cells can be genetically altered to comprise a stably incorporated recombinant gene (or a nucleic acid sequence) that encodes a desired nucleic acid product and is under myoblast-specific transcription control. Muscle cells genetically altered in this way (recombinant muscle cells) can then be examined for expression of the recombinant gene (or nucleic acid sequence) prior to administration to a mammal. For example, the amount of desired nucleic acid product expressed can be measured according to standard methods (e.g., immunoprecipitation). In this manner, it can be determined in vitro whether a desired nucleic acid product is expressed to a suitable level in muscle cells prior to administration to a mammal. Genetically altered muscle cells (recombinant muscle cells) expressing the desired nucleic acid product to a suitable level can be expanded (grown) for introduction into the circulation of a mammal. Methods for expanding (growing) cells are well known in the art. As discussed above, in a particular embodiment, muscle cells are purified from a donor matched for immunocompatibility with the recipient mammal. Preferably, the donor and recipient are matched for their compatibility for the MHC (HLA) class I (A, B, C) and class II (DR, DQ, DRW) antigens.

### Cellular Compositions

Compositions of the invention include pharmaceutical compositions comprising cells of this disclosure. Administration can be autologous or heterologous. For example, cells can be obtained from one subject, and administered to the same subject or to a different, compatible subject.

Myoblasts of the invention can be administered as therapeutic compositions (e.g., a pharmaceutical composition). Generally, such cellular compositions are formulated in a unit dosage injectable form.

Cellular compositions as described herein can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise a carrier, which can be a solvent or dispersing medium containing, for example, water, saline, phosphate buffered saline, polyol (for example, glycerol, propylene glycol, liquid polyethylene glycol, and the like), and suitable mixtures thereof.

Sterile injectable solutions can be prepared by incorporating the cells described herein in a sufficient amount of an appropriate solvent. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at a selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected and the amount of the agent used. The choice of suitable carriers and other additives will depend on the exact route of administration and the nature of the particular dosage form (e.g., a liquid dosage form can be formulated into a solution, a suspension, a gel, or another liquid form, such as a time release formulation or liquid-filled form).

One consideration concerning the therapeutic use of cells of the invention is the quantity of cells necessary to achieve an optimal effect. The quantity of cells to be administered will vary for the subject being treated. In a preferred embodiment, between 10⁴ to 10⁸ cells, and more preferably 10⁵ to 10⁷ cells, are administered to a subject.

The skilled artisan can readily determine the amounts of cells and optional additives, vehicles, and/or carrier in compositions to be administered. In one embodiment any additive (in addition to the cell(s)) is present in an amount of 0.001% to 50% (weight) solution in phosphate buffered saline, and the active ingredient is present in the order of micrograms to milligrams, such as about 0.0001% to about 5 wt %. In another embodiment, the active ingredient is present at about 0.0001% to about 1 wt %. In yet another embodiment, the active ingredient is present at about 0.0001% to about 0.05 wt %. In still other embodiments, the active ingredient is present at about 0.001% to about 20 wt %. In some embodiments, the active ingredient is present at about 0.01% to about 10 wt %. In another embodiment, the active ingredient is present at about 0.05% to about 5 wt %. For any composition to be administered to an animal or human, and for any particular method of administration, toxicity can be determined by measuring the lethal dose (LD) and LD5o in a suitable animal model e.g., a rodent such as mouse. The dosage of the composition(s), concentration of components therein, and timing of administering the composition(s), which elicit a suitable response can also be determined. Such determinations do not require undue experimentation in light of the knowledge of the skilled artisan, this disclosure, and the documents cited herein. The time for sequential administrations can also be ascertained without undue experimentation.

### Inhibitory Nucleic Acids

Inhibitory nucleic acid molecules are oligonucleotides that inhibit the expression or activity of a target gene. Such oligonucleotides include single and double stranded nucleic acid molecules (e.g., DNA, RNA, and analogs thereof) that bind a nucleic acid molecule encoding a polypeptide that inhibits muscle fusion or extravasation (e.g., antisense molecules, siRNA, and shRNA) as well as nucleic acid molecules that bind directly to the polypeptide to modulate its biological activity (e.g., aptamers). In one embodiment, the inhibitory nucleic acid molecule inhibits the expression of a gene of Table 1, thereby enhancing extravasation or enhancing fusion.

siRNA

Short interfering RNAs (siRNAs) are double-stranded RNA oligomers comprising 21 to 25 nucleotides that effectively down-regulate gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

The nucleic acid sequence of a gene can be used to design siRNAs that can inactivate a target gene. Such siRNAs can be administered directly to an affected tissue or administered systemically. The siRNAs may be used, for example, as therapeutics to treat a muscle disease.

The inhibitory nucleic acid molecules of the present invention may be employed as double-stranded RNAs for RNA interference (RNAi)-mediated knock-down of expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells. In one embodiment of the present disclosure, expression of a gene listed in Table 2 or 3 is reduced in a skeletal muscle cell via an siRNA or an RNAi methodology.

In one embodiment of the disclosure, a double-stranded RNA (dsRNA) oligomer includes between eight and nineteen consecutive nucleobases. The dsRNA can be two distinct strands of RNA that interact sufficiently to form a duplex, or a single RNA strand that has interacted with itself such that a partial duplex results and a small hairpin RNA (shRNA) is formed. Typically, dsRNAs are about 21 or 22 base pairs, but may be shorter or longer (up to about 29 nucleobases). dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

shRNAs comprise an RNA sequence having a stem-loop structure. A “stem-loop structure” refers to a nucleic acid having a secondary structure that includes a region of nucleotides known or predicted to form a double strand or duplex (stem portion) that is linked on one side by a region of predominantly single-stranded nucleotides (loop portion). The term “hairpin” is also used herein to refer to stem-loop structures. Such structures are well-known in the art. As is known in the art, the secondary structure does not require exact base-pairing. Thus, the stem can include one or more base mispairings or bulges. Alternatively, the base-pairing may not include any mispairings between the strands. The multiple stem-loop structures can be linked to one another through a linker, such as, for example, a nucleic acid linker, a microRNA (miRNA) flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. The term “shRNA” also includes miRNA embedded shRNAs (miRNA-based shRNAs), wherein the guide strand and the passenger strand of the miRNA duplex are incorporated into an existing (natural) miRNA or into a modified or synthetic (designed) miRNA. In some instances, the precursor miRNA molecule can include more than one stem-loop structure. MicroRNAs are endogenously encoded RNA molecules approximately 22 nucleotides long and generally expressed in a highly tissue- or developmental-stage-specific fashion. More than 200 distinct miRNAs have been identified in plants and animals. These small regulatory RNAs are believed to serve important biological functions by two prevailing modes of action: (1) by repressing the translation of target mRNAs, and (2) RNA interference (RNAi)-mediated cleavage and degradation of mRNAs. In the latter case, miRNAs function siRNAs. One can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and vectors that allow for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells by any means known in the art. A producer cell line generates infectious retroviral vector particles, which include a polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence as described herein can be used to inhibit expression of a nucleic acid molecule in vivo (e.g., a nucleic acid molecule listed in Table 2 or 3). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly one embodiment of the present disclosure features a catalytic RNA molecule that includes, in the binding arm, an antisense RNA having between eight and nineteen consecutive nucleobases. In some embodiments of this disclsoure, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting and those skilled in the art will recognize that an enzymatic nucleic acid molecule of this disclosure has a specific substrate binding site complementary to one or more of the target gene RNA regions. Those skilled in the art will also recognize that these nucleotide sequences within or surrounding the substrate binding site contribute to the molecule's RNA cleaving activity.

Essentially any method for introducing a nucleic acid construct into cells can be employed. Physical methods of introducing nucleic acids include injecting a solution containing the construct: bombarding cells with particles covered by the construct; soaking a cell, tissue sample, or organism in a solution of the nucleic acid; or electroporation of cell membranes in the presence of the construct. A viral construct packaged into a viral particle can be used to accomplish efficient introduction of an expression construct encoding an shRNA into a cell and transcription of the encoded shRNA. Other methods known in the art for introducing nucleic acids to cells can be used, such as lipid-mediated carrier transport and chemical mediated transport, such as calcium phosphate, and the like. Thus, the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors(e.g., plasmid vectors) comprising either an RNA polymerase II or RNA polymerase III promoter can be employed. Expression of endogenous miRNAs is controlled by RNA polymerase II (Pol II) promoters, and in some cases, shRNAs are more efficiently driven by Pol II promoters, as compared to RNA polymerase III promoters (Dickins et al., 2005, Nat. Genet. 39: 914-921). In some embodiments, expression of shRNA can be controlled by an inducible promoter or a conditional expression system, including, without limitation, RNA polymerase type II promoters. Examples of useful promoters in the context of the present disclosure are tetracycline-inducible promoters (including TRE-tight), Isopropyl β-D-1-thiogalactopyranoside (IPTG)-inducible promoters, tetracycline transactivator systems, and reverse tetracycline transactivator (rtTA) systems. Constitutive promoters can also be used, as can cell- or tissue-specific promoters. Many promoters are ubiquitous as they are expressed in all cell and tissue types. Some embodiments use tetracycline-responsive promoters, one of the most effective conditional gene expression systems, in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Kits

Cells of the invention may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine if a consistent result is achieved.

### Administration of Cellular Compositions

Compositions comprising cells of the present disclosure can be administered to (introduced into) a mammal according to methods known to those practiced in the art. In one embodiment, the cells are administered systemically by injection. Other modes of administration (parenteral, mucosal, implant, intraperitoneal, intradermal, transdermal (e.g., in slow release polymers), intramuscular, intravenous including infusion and/or bolus injection, and subcutaneous) are generally known in the art. In some embodiments, muscle cells are administered in a medium suitable for injection, such as phosphate buffered saline, into a mammal.

The purified muscle cells used in the methods of the present invention can be obtained from a mammal to whom they will be returned or from another/different mammal of the same or different species (donor) and introduced into a recipient mammal. For example, the cells can be obtained from a pig and administered to a human. In an embodiment of particular interest, the recipient mammal is a human patient.

The present invention provides methods of treating diseases and/or disorders or symptoms thereof that comprise administering a therapeutically effective amount of a pharmaceutical composition comprising a cell of the invention to a subject (e.g., a mammal, such as a human). Thus, one embodiment is a method of treating a subject suffering from or susceptible to a muscle disease or disorder or symptom thereof. The method includes administering to the mammal a therapeutic amount of cells described herein sufficient to treat the disease or disorder or symptom thereof, under conditions such that the disease or disorder is treated.

The methods herein include administering to the subject (including a subject identified as in need of such treatment) an effective amount of a cell described herein, or a composition described herein to produce such effect. A health care professional can rely on her judgment to form a subject opinion of whether a subject is in need of such treatment. Alternatively, objective standards (e.g. measurable by a test or diagnostic method) can be used to identify such a subject.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject who does not have, but is at risk of or susceptible to developing, a disorder or condition.

The therapeutic methods of the invention (which include prophylactic treatment) in general comprise administration of a therapeutically effective amount of a compound or formulae disclosed herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

Another embodiment provides a method of monitoring treatment progress. The method includes a step of determining a level of a diagnostic marker (e.g., any target delineated herein modulated by a compound herein or diagnostic measurement (e.g., a screen or assay) in a subject suffering from or susceptible to a disorder or symptoms thereof associated with muscle disease, in which the subject has been administered a therapeutic amount of a compound disclosed herein sufficient to treat the disease or symptoms thereof. The level of a marker determined in the method can be compared to known levels of the marker in either healthy normal controls or in other afflicted patients to establish the subject's disease status. In some embodiments, a second level of the marker in the subject is determined at a time point later than the determination of the first level, and the two levels are compared to monitor the course of disease or the efficacy of the therapy. In some embodiments, a pre-treatment level of the marker in the subject is determined prior to beginning treatment according to this disclosure; this pre-treatment level of the marker can then be compared to the level of the marker in the subject after the treatment commences to determine the efficacy of the treatment.

### CRISPR Screening

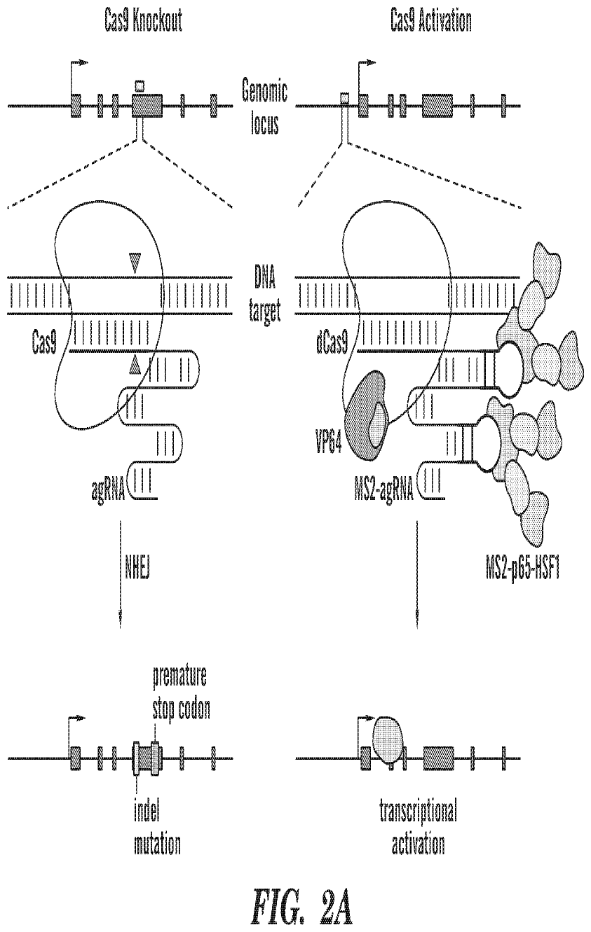

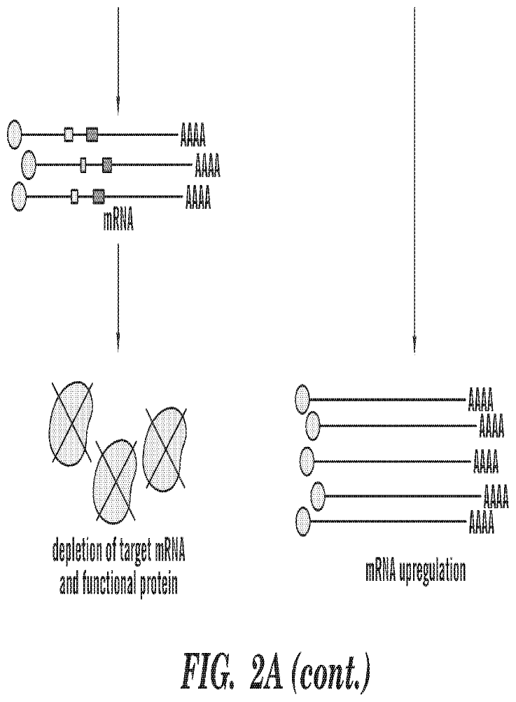

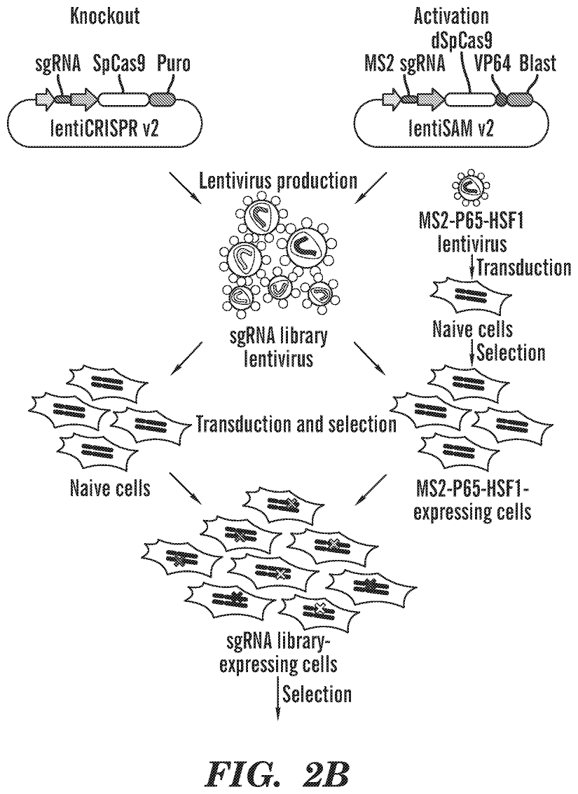

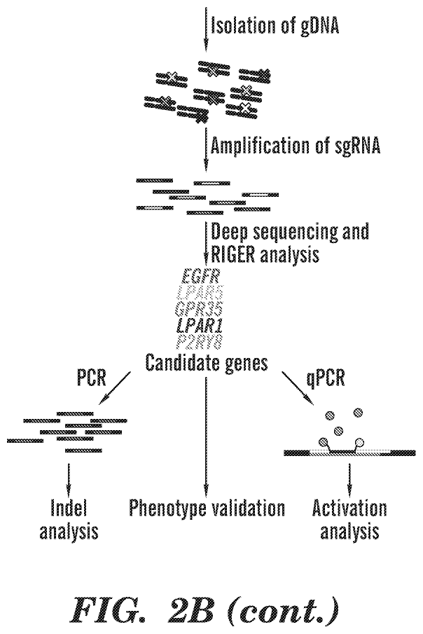

The emergence of CRISPR (clustered regularly interspaced short palindromic repeats) gene editing technology has enabled the systematic interrogation of gene function on a genome-wide scale (Shalem et al. Science (New York, N.Y.) 343 (6166): 84-87 2014). Loss- or gain-of-function (LoF and GoF, respectively) perturbations across the entire genome have recently been made possible by way of incorporating Cas9 endonuclease from the microbial immune system CRISPR with single guide RNA (sgRNA) libraries to induce precise DNA modification at targeted sites (Cong, et al. 2013, Science 339 (6121): 819-23). When combined with efficient lentiviral delivery, genome-scale CRISPR-Cas9 editing platforms provide a powerful strategy to perform LoF and GoF screens to elucidate gene function (Miles et al., FEBS 283: 3170-80 2016). In LoF screens, Cas9 is employed to generate a double-stranded break at a precise target locus, triggering an error-prone repair mechanism that introduces frameshift indels and ultimately leads to LoF mutations (FIG. 2A, left). The use of CRISPR LoF libraries has thus far been used to determine essential genes involved in drug resistance (Shalem et al. 2014, supra) and cancer metastasis (Chen et al. Cell 160 (6) Elsevier Inc.: 1246-60 2015). The LoF Gecko library utilized in our loss-of-function resistance screen consisted of 123,411 sgRNAs to target 19,050 coding genes (6 different guides per gene). GoF screens in comparison are more complex, requiring several engineered components such as a fusion complex involving an inactivated Cas9 and a transcriptional activator (Cas9-VP64), and modified sgRNAs that incorporate MS2 bacteriophage coat proteins, enabling it to recruit two other activation domains (p65 and HSF-1) to the Cas9-VP64 complex (FIG. 2A, right) (Konermann et al. 2014 et al. Nature 517 (7536): 583-88). Thus, the synergistic activation mediator (SAM) complex utilizes three different transcriptional effectors to achieve transcriptional up-regulation. The SAM library proposed for use in our GoF screen includes 70,290 unique sgRNAs designed to up-regulate expression of 23,430 coding genes (three different guides per gene). These sgRNA target sites are spread across the proximal promoter and the transcriptional start site of each gene, and have been demonstrated to up-regulate gene expression anywhere between two- to fifteen-fold. Both LoF and GoF plasmid libraries are commercially available through Addgene. Pooled libraries must be packaged into lentivirus particles and titered such that most cells are transduced with only one stably integrating sgRNA. Screening strategy is outlined in FIG. 2B

The present disclosure requires, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Results from Fusion Screen

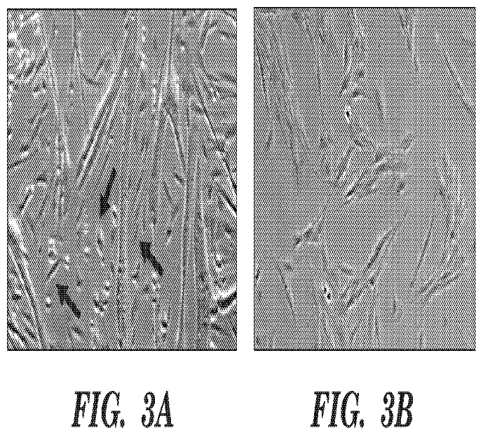

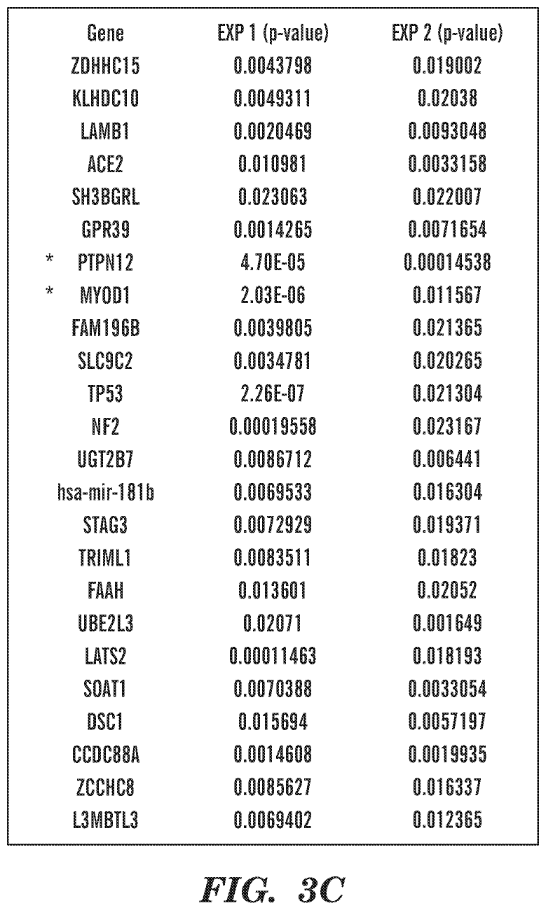

The CRISPR-Cas9 knock-out library was successfully employed to perform genome-wide loss-of-function screens to identify genes that when knocked-out prevented fusion of mononuclear myoblasts into multinucleated myotubes. These screens identified fusion-defective cell populations grown in the presence of low serum differentiation medium (FIG. 3A). Downstream sequencing and enrichment analysis was performed to identify the CRISPR knock-out target in these cells. The reproducibility of these genes across multiple independent screens strengthens confidence in their biological significance in the context of this assay (FIG. 3C). sgRNAs targeting the MYOD1 gene, a known regulator of muscle cell differentiation, showed significance in both screens. Similarly, sgRNAs targeting the PTPN12 gene, a known regulator of macrophage fusion, was also significantly enriched in the fusion-defective population. Both of these gene hits provide proof of principle that the methodology can identify genes involved in promoting cell fusion.

### Example 2

### Achieving Genome-Wide Knock-Out or Over-Expression of Human Genes Using CRISPR Libraries

Both loss- and gain-of-function screens were performed using commercially available CRISPR libraries from AddGene comprising of pooled sgRNA lentivirus constructs. For the activation library, CRISPR-induced transcriptional activation requires additional components for the formation of the SAM complex—an inactive Cas9 fused to a transcriptional activation domain (dCas9-VP64) and effector components (MS2-p65-HSF1) (FIG. 2A). Lentivirus constructs for each component of the complex must be amplified and packaged into lentivirus particles. Prior to transduction with the pooled sgRNA library, a population of cells was established with stable integration of both dCas9-VP64 and MS2-p65-HSF1 constructs that have been transduced and undergone antibiotic selection. This population (or unmodified myoblasts for LoF screen) was transduced with the sgRNA activation library at a low multiplicity of infection (MOI) of 0.3 to reduce the probability of cells taking up more than one sgRNA. Transduction of 300 cells per guide RNA is optimal with this representation maintained over the 7-day antibiotic selection process. See FIG. 2B for experimental flow diagram.

### Example 3

### Separation of Mononuclear Myoblasts and Multinuclear Myotubes

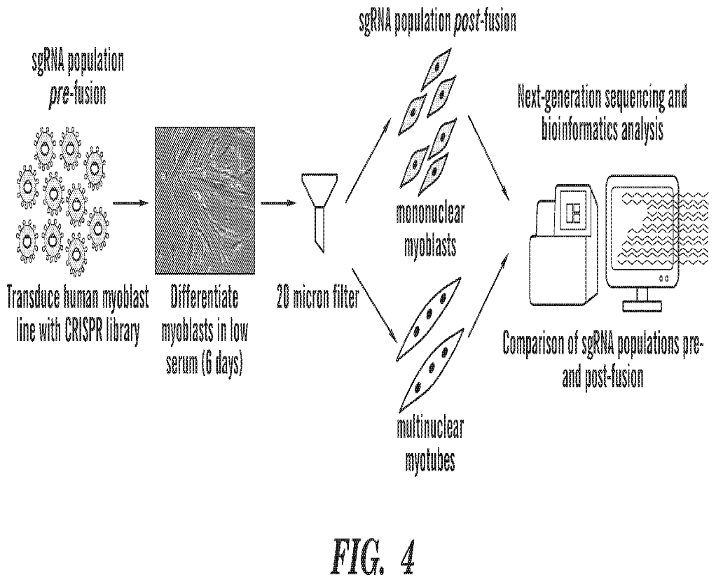

A minimum of 40 million gene-edited cells were plated at high density in differentiation medium without serum. After six days, cells have either fused to form multinucleated myotubes or have not fused and remain as mononuclear myoblasts (FIG. 4). Cells were detached from their substratum and passed through a 20 μm filter to separate the mononuclear and multinuclear fractions. Both fractions were snap frozen for genomic DNA extraction. Gene-edited cells were switched to media to promote myogenic fusion into multinucleated myotubes. Cells that failed to fuse and remained mononuclear were separated by filtration and sequenced to identify their CRISPR gene modification. This approach identified the genes identified in Table 1.

| | | | | | | | | |
|---|---|---|---|---|---|---|---|---|
| | Exp1 | Exp1 | Exp2 | Exp2 | Exp3 | Exp3 | Exp4 | Exp4 |
| Gene | p-val | sgRNA | p-val | sgRNA | p-val | sgRNA | p-val | sgRNA |

Top Hits from 4 Myoblast Experiments

| | | | | | | | | |
|---|---|---|---|---|---|---|---|---|
| UBE2L3 | 0.02071 | 4 | 0.001649 | 5 | 0.0023426 | 4 | 0.00021457 | 5 |
| MYOD1 | 2.03E−06 | 5 | 0.011567 | 5 | 1.13E−06 | 5 | 0.014349 | 5 |

[symbol]

om 3 Myoblast Experiments

| | | | | | | | | |
|---|---|---|---|---|---|---|---|---|
| MYOD1 | 2.03E−06 | 5 | 0.011567 | 5 | 1.13E−06 | 5 | 0.014349 | 5 |
| KRTAP9-3 | 0.83736 | 1 | 0.018523 | 3 | 0.0109 | 5 | 0.0037996 | 3 |
| TRIM24 | 0.30461 | 2 | 0.0052205 | 4 | 0.011925 | 4 | 0.015631 | 4 |
| PLAC1L | 0.66784 | 2 | 0.014127 | 4 | 0.010755 | 4 | 0.0079224 | 4 |
| TP53 | 2.26E−07 | 6 | 0.021304 | 2 | 0.0032041 | 4 | 0.1283 | 2 |
| RAX2 | 0.11745 | 4 | 0.017812 | 2 | 0.0075357 | 1 | 0.0068534 | 2 |
| KEAP1 | 0.016417 | 4 | 0.13754 | 2 | 1.88E−05 | 5 | 0.0011913 | 6 |
| GPR39 | 0.0014265 | 5 | 0.0071654 | 2 | 0.50579 | 3 | 0.024378 | 3 |
| hsa-mir- | 0.0069533 | 4 | 0.016304 | 4 | 0.015423 | 2 | 0.47425 | 1 |
| 181b-1 | | | | | | | | |
| SOAT1 | 0.0070388 | 4 | 0.0033054 | 4 | 0.95435 | 0 | 0.0053846 | 4 |
| N4BP1 | 1.58E−06 | 6 | 0.035573 | 3 | 0.0004678 | 5 | 0.012097 | 4 |
| SOX11 | 0.029705 | 3 | 0.018144 | 4 | 0.0023268 | 4 | 0.00856 | 4 |
| ZDHHC15 | 0.0043798 | 1 | 0.019002 | 3 | 0.82988 | 1 | 0.019861 | 3 |
| UGT2B7 | 0.0086712 | 5 | 0.006441 | 3 | 0.73178 | 2 | 0.0069895 | 3 |
| DYRK1A | 0.0033176 | 5 | 0.066086 | 3 | 4.30E−06 | 6 | 0.019505 | 5 |
| CCDC88A | 0.0014608 | 5 | 0.0019935 | 4 | 0.58913 | 2 | 0.0049957 | 4 |
| NF2 | 0.00019558 | 5 | 0.023167 | 5 | 1.13E−06 | 4 | 0.77318 | 2 |
| LATS2 | 0.00011463 | 4 | 0.018193 | 5 | 0.0036454 | 5 | 0.7865 | 1 |
| DSC1 | 0.015694 | 5 | 0.0057197 | 4 | 0.46155 | 2 | 0.0053344 | 5 |
| UBE2L3 | 0.02071 | 4 | 0.001649 | 5 | 0.0023426 | 4 | 0.00021457 | 5 |
| ACE2 | 0.010981 | 3 | 0.0033158 | 4 | 0.62337 | 1 | 0.013511 | 4 |

[symbol]

From Myotubes

| | Exp2 | Exp2 | Exp3 | Exp3 | Exp4 | Exp4 |
|---|---|---|---|---|---|---|
| Gene | p-val | sgRNA | p-val | sgRNA | p-val | sgRNA |
| UBE2L3 | 0.82088 | 1 | 0.39082 | 1 | 0.35954 | 3 |
| CCDC88A | 0.17679 | 3 | 0.10907 | 3 | 0.075234 | 4 |
| SOAT1 | 0.079107 | 3 | 0.42979 | 3 | 0.080652 | 3 |
| TP53 | 0.011799 | 3 | 1.02E−05 | 4 | 0.13095 | 2 |
| hsa-mir- | 0.08792 | 1 | 0.53247 | 1 | 0.37736 | 2 |
| 181b-1 | | | | | | |
| SOX11 | 0.78027 | 1 | 0.74573 | 1 | 0.84886 | 1 |
| ACE2 | 0.019081 | 3 | 0.23827 | 3 | 0.10117 | 4 |
| UGT2B7 | 0.15753 | 3 | 0.23122 | 2 | 0.05295 | 2 |
| TRIM24 | 0.0041198 | 4 | 0.7623 | 1 | 0.00033486 | 3 |
| DYRK1A | 0.15443 | 2 | 0.18366 | 4 | 0.15076 | 3 |
| N4BP1 | 0.23862 | 3 | 0.81622 | 1 | 0.11781 | 2 |
| NF2 | 0.46837 | 2 | 1.47E−05 | 3 | 0.97673 | 0 |
| PLAC1L | 0.036733 | 4 | 0.023212 | 4 | 0.22438 | 4 |
| ZDHHC15 | 0.051511 | 4 | 0.86044 | 1 | 0.024643 | 3 |
| KRTAP9-3 | 0.0019357 | 3 | 0.035195 | 3 | 0.0085509 | 4 |
| LATS2 | 0.03734 | 4 | 0.0021224 | 4 | 0.5822 | 3 |
| KEAP1 | 0.72292 | 1 | 0.40823 | 3 | 0.62327 | 2 |
| GPR39 | 0.23207 | 1 | 0.16527 | 4 | 0.38537 | 2 |
| DSC1 | 0.19581 | 3 | 0.11901 | 3 | 0.068017 | 5 |
| MYOD1 | 0.35202 | 1 | 0.7157 | 1 | 0.55289 | 1 |
| RAX2 | 0.67051 | 1 | 0.52696 | 1 | 0.31431 | 2 |

[symbol]

indicates data missing or illegible when filed

Genes listed in Table 1 may optionally be characterized as described in Examples 4 and 5.

### Example 4

### Systemic Injection of Gene-Edited Myogenic Cells

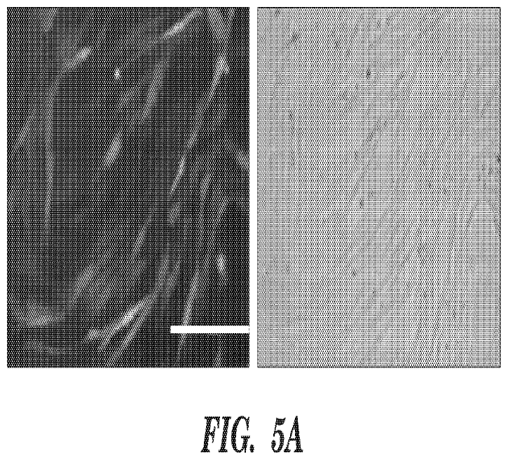

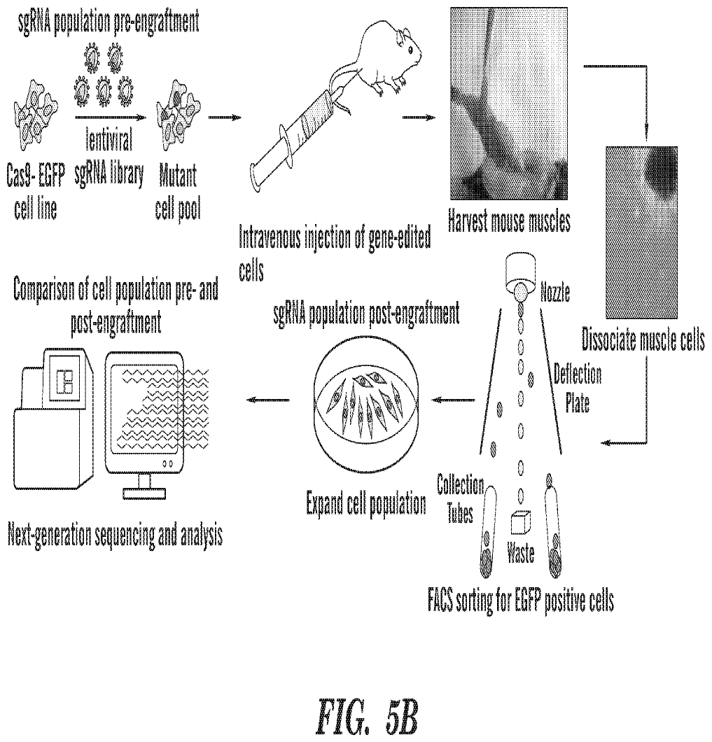

Transduction of myogenic cells prior to transplantation was performed on a GFP-expressing myogenic line to enable determination of the fate of cells post-intravenous injection (FIG. 5A). A minimum of 40 million gene-edited GFP-expressing myogenic cells are collected in PBS and injected into the tail vein of NOD-Rag1 null mdx^{5cv} mice (FIG. 5B). The next day, injected mice are sacrificed and muscle groups extracted and digested with dispase and collagenase. Digested muscle is passed through a filter and incubated with lysis buffer. Cell suspension is prepared for fluorescent activated cell sorting to separate GFP-positive cells that have successfully extravasated from the circulatory system. GFP-positive cells are cultured until confluence and prepared for next-generation sequencing to identify CRISPR gene-edit.

### Example 5

### Next-Generation Sequencing to Identify Enriched Genes for Fusion or Engraftment

To identify pro-fusion factors, genomic DNA is isolated from three populations of cells for gene enrichment analysis: 1) a population of gene-edited cells prior to differentiation; 2) a population of gene-edited cells that remain mononuclear post-differentiation; and 3) a population of gene-edited cells that are multinuclear post-differentiation. A PCR reaction is performed to amplify sgRNA sequences from each population and to attach experimental barcodes and Illumina sequencing primers. The resulting amplicons are sequenced using the Illumina Next-seq platform using the Broad Institute's Walk-up Sequencing Service. Sequencing data is processed using the open-source software MaGeCK (Li et al. 2014) to statistically normalize reads between the three populations of cells to be compared (pre- and post-differentiation). Next, an alignment of sgRNA reads is performed against the library to identify genes having significantly enriched frequencies post-differentiation. Genes enriched via more than one sgRNA are used to confirm true positive hits.

To identify pro-engraftment factors, genomic DNA is isolated from two populations of cells for gene enrichment analysis: 1) a population of gene-edited cells prior to intravenous injection into mouse; 2) a population of gene-edited cells that have successfully extravasated from circulation and have homed into skeletal muscle tissues. The same downstream PCR reactions are performed as described for the procedure to identify pro-fusion factors in preparation for next-generation sequencing. Follow-up bioinformatic analyses will identify sgRNA gene targets enriched in rare extravasated cell populations.

Genes identified in Table 1 are modulated to increase or decrease their expression. In one embodiment, genes identified in Table 1 are over-expressed in healthy muscle cells, which are then prepared for infusion into a subject suffering from muscular dystrophy where the infused cells replace diseased muscles with new healthy muscles. In another embodiment, genes identified in Table 1 are targeted for knock-down or knock-out in healthy muscle cells (e.g., using an inhibitory nucleic acid molecule) and then prepared for infusion into a subject suffering from muscular dystrophy where the infused cells replace diseased muscles with new healthy muscles. The methods of the invention can be used in conjunction with known methods of cell transplantation whose efficacy has been limited by a failure of the transplanted cells to extravasate and/or fuse with endogenous muscle cells.

Genes identified in Table 1 are also useful for the identification of agents (e.g., small molecules, polypeptides, polynucleotides) that potentially mimic the knock-down or over-expression of the pathways and/or genes of interest. Individual cell knock-out or over-expression models of the genes listed in Table 1 were generated.

Pro-engraftment and pro-fusion factors are used to engineer a corrective muscle cell with these genetic switches; or alternately identify small molecules that mimic the function of these switches and can be delivered alongside the therapeutic cells. To ultimately assess therapeutic capacity, the cells are delivered systemically as a highly engraftable myogenic population to NOD-Rag1 null mdx^{5cv} mice, which are then assessed for improved muscle structure and function.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An isolated cell lacking a gene of Table 1 or expressing a reduced level of a gene of Table 1.

2. The isolated cell of claim 1, wherein the cell comprises a CRISPR (clustered regularly interspaced short palindromic repeats)-edited genome or expresses an inhibitory nucleic acid molecule targeting a gene of Table 1.

3. The isolated cell of claim 2, wherein the inhibitory nucleic acid molecule is an antisense oligonucleotide molecule, a short interfering RNA (siRNA) molecule, or an small hairpin (shRNA) molecule.

4. An isolated cell comprising an expression vector encoding a gene of Table 1.

5. The isolated cell of claim 4, wherein the cell is a genetically engineered cell derived from a subject in need of cell transplantation therapy.

6. The isolated cell of claim 4, wherein the cell is a healthy cell.

7. The isolated cell of claim 4, wherein the cell is a muscle cell or cancer cell.

8. A pharmaceutical composition comprising a cell of claim 7 in a pharmaceutically acceptable excipient.

9. A method for treating a subject in need of muscle cell transplantation, the method comprising administering to the subject a muscle cell of claim 7.

10. A method for identifying a gene required for cell fusion, the method comprising
a) editing each gene in a genome of a cell population using a clustered regularly interspaced short palindromic repeats (CRISPR) library;
b) growing the cell population under conditions that permit cell fusion;
c) isolating mononucleate cells that are fusion defective; and
d) identifying single guide RNAs (sgRNAs) that are enriched in fusion defective cells.

11. A method for identifying a gene that promotes extravasation, the method comprising
a) editing each gene in a genome of a cell population using a clustered regularly interspaced short palindromic repeats (CRISPR) library, wherein each cell of the cell population comprises a detectable reporter;
b) administering the cell population to a non-human mammal;
c) isolating cells comprising the detectable reporter from the mammal; and
d) sequencing the genomes of the cells to identify the CRISPR gene edit in each cell, thereby identifying a gene required for extravasation.

12. The method of claim 10, wherein the library is a knock-out or upregulation library.

13. A method of identifying an agent that mimics the effect of an edited gene, the method comprising carrying out the method of claim 9 in the presence of the agent.
